(19) United States
(12) Patent Application Publication
(10) Pub. No.: US 2026/0183325 A1
(43) Pub. Date: Jul. 2, 2026

(54) SUSTAINED OR IMMEDIATE RELEASE ENERGY, MENTAL FOCUS, AND PHYSICAL RECOVERY NUTRITIONAL SUPPLEMENT BLEND, DIETARY SUPPLEMENT, FUNCTIONAL FOOD AND FUNCTIONAL BEVERAGE

(71) Applicant: Matchstick Brands, LLC, Pewee Valley, KY (US)

(72) Inventors: Michael Thomas Macchiarulo, Pewee Valley, KY (US); Lynda M. Doyle, Amenia, NY (US); Mark Macchiarulo, Pewee Valley, KY (US); Crystal D. Webber, Pittsburgh, PA (US)

(21) Appl. No.: 19/545,704

(22) Filed: Feb. 20, 2026

Related U.S. Application Data
(63) Continuation of application No. PCT/US2024/044972, filed on Sep. 3, 2024.
(60) Provisional application No. 63/580,517, filed on Sep. 5, 2023.

(51) Int. Cl. A61K 31/7068; A61K 31/522; A61K 36/736; A61K 36/9062; A61P 25/28

(52) U.S. Cl. CPC A61K 31/7068; A61K 31/522; A61K 36/736; A61K 36/9062; A61P 25/28

(57) ABSTRACT

Provided herein are methods and compositions for Sustained Release Energy, Mental Focus, and Physical Recovery Nutritional Supplement Blend, Dietary Supplement, Functional Food and Functional Beverage.

### CROSS-REFERENCE TO RELATED APPLICATIONS

The present application claims priority to PCT application serial no. PCT/US2024/044972, filed Sep. 3, 2024, which claims priority to U.S. Provisional application Ser. No. 63/580,517, filed Sep. 5, 2023, each herein incorporated by reference in their entireties.

### BACKGROUND

This invention relates to specialty nutraceuticals and botanical extracts to provide health benefits for active individuals, more specifically to a combination specifically outlined below to promote rapid on-set and long-lasting energy, mental and physical performance, recovery, nootropic and antioxidant support.

Caffeine is known to cause jitters, heart palpitations, anxiety and nervousness, as well as have detrimental effects at higher doses. The adverse events of caffeine may be minimized with continual low doses over the day, but this is inconvenient, or the use of controlled-release product.

The present invention attempts to solve these problems, as well as others.

### SUMMARY OF THE INVENTION

Provided herein are methods and compositions for Sustained Release Energy, Mental Focus, and Physical Recovery Nutritional Supplement Blend, Dietary Supplement, Functional Food and Functional Beverage.

The methods and compositions are set forth in part in the description which follows, and in part will be obvious from the description, or can be learned by practice of the methods and compositions. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.,” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DESCRIPTION OF EMBODIMENTS

Generally speaking, the composition or formulation includes at least three bioactives, at least one bioactive which is in a controlled-release format or immediate release format to exert its effects and support the other two bioactive both immediately and over time. In one embodiment, the at least one bioactive includes at least one antioxidant. The composition or formulation includes a controlled release or immediate release of caffeine over a period of about six plus hours to provide the benefits of caffeine without the negative effects. The composition or formulation includes antioxidant support from caffeine and/or the other ingredients both immediately and over an extended period. In one embodiment, caffeine is substituted with a caffeine derivative, a caffeine modifier, or a caffeine substitute including Alpinia galanga.

In one embodiment, the composition or formulation is to be ingested as a dietary supplement, functional food or functional beverage. The composition or formulation comprises the combination of an effective amount of caffeine, citicoline, and tart cherry extract. In the present disclosure, an “effective amount” or an “effective dose” of caffeine, tart cherry, citicoline and combinations thereof, or composition refers to an amount of caffeine, tart cherry, citicoline that, once administered to a subject, will reach the subject's bloodstream and/or bodily tissues.

The composition or formulation comprises the combination of an effective amount of caffeine, citicoline, and tart cherry extract to provide a synergistic effect of caffeine with negative side effects and increased energy for the subject. The term “synergistic” as used herein refers to the phenomenon wherein the cumulative pharmacological effect of two or more ingredients when used in combination is higher than the sum of the effect of each of them tested individually. Synergism plant derivatives exert their potential when administered with conventional drugs is synergism—a positive interaction that occurs when two compounds are given in combination, resulting in an inhibitory effect greater than the simple sum of the effects of the single drugs administered individually. The term “potentiating” as used herein refers to the phenomenon where the efficacy of an active ingredient is significantly enhanced when it is combined with a second ingredient, wherein said second ingredient itself does not demonstrate any efficacy in the same pharmacological test. In some cases of potentiation, not only is said second ingredient is devoid of the pharmacological effect being measured, but it may also even cause an opposite effect, when assayed alone. An example of such a case would be as follows: ingredient A is anti-energy; ingredient B is pro-energy; when A and B are combined, said combination produces an energy effect that is greater than seen with B alone. In the context of the present invention, potentiation is regarded as a special case of synergism. Thus, the term ‘synergism’ (or synergistic, or the like), when used to define the properties of a composition of the present invention, also includes within its range of meaning the potentiation effect described immediately hereinabove.

The synergistic effects between plant-derived bioactives and neuro stimulants has been demonstrated by different mechanisms. These effects are generally based on four different mechanisms: the synergistic multitarget effects (when plant derivatives impact on different targets and not only on a single one), the modulation of pharmacokinetic or physicochemical effects (when synergism influences the physicochemical properties, including solubility, of a compound, improving its bioavailability), the interference with resistance mechanisms (when plant derivatives antagonize the onset of drug resistance observed with antibiotics or anticancer drugs against drug-resistant microorganisms or cancer cells), and the elimination or neutralization potential (when a plant derivative is capable of reducing or neutralizing the toxicity of a drug, impacting its adverse effects and improving treatment).

In one embodiment, the composition or formulation comprises the combination of an effective amount of caffeine, citicoline, and tart cherry extract to synergistically provide immediate and sustained for Energy, Nootropic effect, Mental and physical performance, such as focus, Recovery, and/or Antioxidant support.

In one embodiment, caffeine or natural sources of caffeine, including but not limited to guarana (Paullinia cupana), coffee (e.g., Coffea arabica, Coffea Robusta, etc.) and tea (e.g., Camellia sinensis, etc.) and their extracts, etc. (immediate release and, but not limited to Sustained or Extended or Controlled release) between about 1 mg and about 600 mg per serving.

Citicoline (immediate release and, but not limited to Sustained or Extended or Controlled release) between about 1 mg and about 1,000 mg per serving.

Tart Cherry Extract (immediate release and, but not limited to Sustained or Extended or Controlled release) between about 1 mg and about 1000 mg per serving.

The composition or formulation is with or without a controlled release (including sustained or extended release) ingredient comprised of any combination thereof.

### Caffeine

Caffeine or natural sources of caffeine, include, but are not limited to, guarana (Paullina cupana), coffee (e.g., Coffea arabica, Coffea Robusta, etc.) and tea (e.g., Camellia sinensis, etc.) and their extracts, etc. (immediate release and, but not limited to Sustained or Extended or Controlled release) between about 0.1%/wt. to about 99.9%/wt. of the composition.

Caffeine used in the invention can be obtained from various sources, such as cocoa beans, kola nuts, tea leaves, guarana berries, guayusa, and coffee beans. The present invention preferably uses plant-derived caffeine rather than synthetic caffeine. Ideally, caffeine may come from coffee beans. It is envisaged that plant-derived, or “natural” caffeine has a more appealing taste compared to synthetic caffeine, perhaps as a result of small traces of other natural extracts retained during production of natural caffeine. Caffeine levels or ratios given herein refer to the absolute level of caffeine rather than the sum total of caffeine and other components present in natural caffeine extract. For instance, where a product calls for 0.9 wt. % caffeine and the natural caffeine extract itself is 90 wt. % caffeine, the product contains 1.0 wt. % natural caffeine extract.

Alternatively, caffeine may be substituted with Alpinia galanga. Alpinia galanga, a plant in the ginger family, bears a rhizome and is one of four plants known as “galangal”. Its common names include greater galangal, lengkuas, and blue ginger. Alpinia galanga rhizome contains the flavonol galangin. The rhizome contains an oil known as galangol, which upon fractional distillation produces cineol (which has medicinal properties), pinene, and eugenol, among others. Alpinia galanga includes 1,8-cineole, α-fenchyl acetate, camphor, methyl cinnamate, and guaiol.

In other embodiments, caffeine modifiers, derivatives, and metabolites may be included or substituted as the stimulant.

### Citicoline

Citicoline (immediate release and, but not limited to Sustained or Extended or Controlled release) between about 0.1%/wt. and about 99.9%/wt. The molecular formula is C₁₄H₂₅N₄NaO₁₁P₂, where the choline cytosine nucleoside diphosphate is a monosodium salt of choline cytosine nucleoside diphosphate, is a nucleoside derivative, is chemically named as monosodium salt of choline cytosine nucleoside diphosphate and has a citicoline sodium content of not less than 98% in a dried product.

According to one embodiment, citicoline (cytidine-5′-diphosphocholine) used in the compositions or salts thereof such as for example the mono-sodium salt could be purchased or prepared according to the protocols described in the known art such as for example Kyowa production protocol (Drug Master File citicoline Kyowa Haakko Kogyo Co., Ltd). The concentration of citicoline in said compositions preferably is comprised in the range between 5 and 30 mg per gram or ml of composition. According to one embodiment, citicoline is used at a concentration per unit dose between about 0.0035 and about 1.5 mg/unit dose. Under “unit dose” the dose is meant that is administered each time to the patient, both divided into plural administrations during the day, and daily, and at intervals of days. The herein described compositions could comprise, besides citicoline, other active principle for topical use in treating glaucoma like e.g., topical antihypertensive drugs.

In other embodiments, citicoline includes derivatives, substituted derivatives, analogues, prodrugs, and salts thereof. Salts may include counterions (defined as “X−”) selected from chloride, bromide, iodide, and the like. For example, one useful salt is the chloride salt of NR (“NR—Cl”). Further salts may include, but are not limited to, fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfate, tartrate, hydrogen tartrate, malate, hydrogen malate, maleate, fumarate, citrate, stearate, palmitate, myristate, laurate, caprate, caprylate, caproate, oleate, linoleate, sulfonate, trifluoromethanesulfonate, trichloromethanesulfonate, tribromomethanesulfonate, trichloroacetate, tribromoacetate, and trifluoroacetate. Citicoline may be derived from any source.

### Tart Cherry Extract

Tart Cherry Extract includes a formulation for immediate release and, but not limited to Sustained or Extended or Controlled release between about 0.1%/wt. to about 99.9%/wt. In another embodiment of any of the above one or more food powders, the tart cherry extract may be about 1-2 mg/ml polyphenol equivalent.

There is a large and separate body of literature that has studied the use of tart cherries as improving endurance, performance and recovery in athletics. The cultivars used are generally speaking Montmorency cherries, known for high anthocyanin and phenolic levels. A large majority of these studies show significant improvement in endurance performance and recovery and some of these studies are double-blinded placebo-controlled crossover designed. Further evidence of the mechanism is that in the few studies that had negative outcomes, the dosage used was insufficient to lower the uric acid.

There are some studies that have tested tart cherries as improving aspects of metabolic health including a study showing a lowering of insulin resistance. Without being limited by any particular theory, Tart cherry (Montmorency and Balaton cultivars) are known to have the highest levels of phenolics and anthocyanins. However, the anthocyanin I, II, and Phenolic concentrations have not been fully established. High anthocyanin, high polyphenolic Tart cherries such as Montmorency and Balaton inhibit xanthine oxidase (XO) and inhibits COX I and II enzymes that are responsible for generating proinflammatory molecules.

There is a strong relationship between inflammation and cell energetics. The anti-inflammatory component of tart cherries is a positive physiological attribute with respect to energy production and as such can be regarded as having energy benefits beyond other XO inhibitors. As such, tart cherry extract increases energy levels, such as increase in ATP availability or production.

In an embodiment, a tart cherry extract of this invention is in liquid form, or liquid may be removed such that the remaining components are in solid form. In an embodiment, a tart cherry extract of this invention is in powdered form.

In one embodiment, a tart cherry extract of this invention is water-soluble.

In an embodiment, an extract of this invention is a “standardized extract” in which specific components have been identified and are present in a minimum or maximum amount or a specific range so as to render the extract consistent at least with regard to those components from one batch to the next. In an embodiment, an extract, including a standardized extract, of the present invention includes caffeine.

### Antioxidants

In other embodiments, other antioxidants may be included such as polyphenols, flavanols, vegetable sources of antioxidants, botanical sources of antioxidants. In one embodiment, Tart Cherry is substituted with antioxidant including polyphenols from a fruit/vegetable extract or powder.

### Dosage

In one embodiment, the formulation is a single serving formulation to be taken once per day by a subject. Thus, in some embodiments a daily dose of active ingredients is provided in a single serving. In some embodiments one daily dose may be divided among two or more servings to be taken in a day, for example among two or more servings of a liquid beverage or two or more tablets. A daily dose of the formulation can be taken by or administered to a subject in a 24-hour period. In some embodiments, a daily dose of the formulation is taken every day for 1 week to 12 months. In some embodiments, the formulation is taken for 1, 2, 3 or 4 weeks. In some embodiments, the formulation is taken for 2, 3, 4, 5, 6, 7, 8, 9, 10, 11 or 12 months.

In some embodiments, a daily dose of the formulation can be provided in one or more daily servings. In some embodiments, a daily dose is provided in a single serving. In some embodiments, a daily dose may be provided in two or more servings (up to 5 servings in 24 hours). While the size of each serving may vary, the quantity of active ingredients to be consumed will be equal to one serving. In the case of a single serving, where one single serving is intended to be taken per day, the single serving will comprise a complete daily dose. In the case of multiple servings, the quantity of active ingredients will be such that the total amount of active ingredients in the servings to be taken in a single day is equivalent to a daily dose.

The methods described herein may comprise administering daily, or every other day, or once a week, an effective dose of the caffeine, tart cherry, citicoline formulation. In an embodiment, the caffeine, tart cherry, citicoline formulation is administered daily for 1-2 days, 1-3 days, 1-12 days, 4-8 days, 8-12 days, 1-24 days, 4 -24 days, 8-24 days, or 12-24 days, or more, or for another period of time according to the present invention.

### Dietary Supplement

In the present invention, a “dietary supplement” refers to a composition comprising caffeine, tart cherry, citicoline which is administered as an addition to a subject's diet, which is not a natural or conventional food. In an embodiment, a dietary supplement administered to a subject includes an effective amount of caffeine, tart cherry, citicoline, such that the caffeine, tart cherry, citicoline composition enters the body and may be acted upon by the body, and reaches blood and/or tissues and/or cells of the subject's body (in particular the brain) to provide neuroprotection and/or attenuate brain injury from stroke in the subject's brain, and otherwise act for instance as discussed throughout this application. In an embodiment, a dietary supplement containing an effective amount of caffeine, tart cherry, citicoline according to the present invention is administered orally. In an embodiment, a dietary supplement or other composition of this invention is administered daily. In an embodiment, the dietary supplement is administered daily for 1 day, 1-7 days, 1-14 days, 1-30 days, 30 days, 30-60 days, or for another period of time according to the present invention. In one embodiment, a dietary supplement according to this invention may be taken chronically, for instance for several months or a year or years. A dietary supplement may be formulated into various forms, such as a powdered form, and otherwise as discussed throughout this application.

The term “caffeine, tart cherry, citicoline formulation” as used herein refers to a composition which is pharmaceutically acceptable and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. The caffeine, tart cherry, citicoline formulation includes configurational isomers (such as cis and trans isomers) and all optical isomers (such as enantiomers) Isomers and diastereomers), racemic, diastereoisomers and other mixtures of these isomers, as well as solvates, hydrates, isomorphs, polymorphs, tautomers, Ester, salt forms and prodrugs. The term “prodrug” refers to a compound that is a drug precursor, which releases the drug in vivo through some chemical or physiological processes after administration (for example, the prodrug is transformed into the desired drug form when it reaches physiological pH or through the action of enzymes). Exemplary prodrugs release the corresponding free acid upon cleavage, and the hydrolyzable ester-forming residues of the compounds of the present invention.

In the present disclosure, an “effective amount” or an “effective dose” of caffeine, tart cherry, citicoline, or caffeine, tart cherry, citicoline composition refers to an amount caffeine, tart cherry, citicoline that, once administered to a subject, will reach the subject's bloodstream and/or bodily tissues.

### Dosage Form

In one embodiment, the caffeine, tart cherry, citicoline formulation is in the form of capsules that consumers can take orally as a gummy. The term “gummy” as used herein is a term of the art and any gummy composition which provides the necessary texture can be used. Gummy products are chewable and most often classified as candies. The texture of a gummy is in part a result of the presence of a gelating agent such as gelatin, although vegetarian-friendly substitutes as known in the art can be used without impacting on the texture. Particularly preferred gummy compositions of the invention include one or more gelating agents selected from gelatin and/or pectin. The level of gelating agent is not crucial so long as the texture of a gummy is achieved, but typical levels in a composition of gummy piece of the invention are about 1 to about 50 wt. % gelating agents, such as about 2 to about 25 wt. %, especially a level of about 4 to about 10 wt. %. A level of about 6% is particularly favored. This relatively low gelating content is favored to ensure the gummies are compressible. Note that in any gummy, the balance of the weight content of the composition is water. This may be added water or may derive from the fruit juice.

In one embodiment, the caffeine, tart cherry, citicoline formulation is in the form of capsules that consumers can take orally. The formulation may also include a powder mixed into liquid or consumed out of packaging as powder. The caffeine, tart cherry, citicoline formulation is free from any contaminants, including foreign material, pesticides, bacterial pathogens, molds, residual solvents, heavy metals and toxins. A certificate of analysis (COA) documented the analytical tests for caffeine, tart cherry, citicoline formulation.

In some embodiments, the formulation is provided in the form of a liquid drink. The liquid drink may comprise the active ingredients in a water base, or in another base liquid that is inert with respect to the active ingredients. The liquid drink may comprise one or more additional inactive ingredients. In some embodiment the liquid drink may comprise one or more gelling or stabilizing agents including, but not limited to xanthan gum, guar gum, propylene glycol, acacia gum and maltodextrin. In some embodiments the liquid drink may comprise one or more colorants, for example a natural color such as carmine or beet root. In some embodiments the liquid drink may comprise one or more preservatives such as citrus oil, sorbates and benzoates. The liquid drink may also comprise one or more flavors such as fruit (e.g., strawberry, melon, apple, peach, lemon, orange, mango, and the like) and non-fruit flavors. In some embodiments the flavor is a natural flavor. In some embodiments the liquid drink may comprise artificial and natural sweeteners.

The caffeine, tart cherry, citicoline formulation of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the caffeine, tart cherry, citicoline formulation and not deleterious to the user. In accordance with one embodiment, suitable nutraceutically acceptable carriers can include oils, plant-based oils, Medium-chain triglyceride (MCT) oil, coconut oil, palm kernel oil, Hemp seed oil, Olive oil, Avocado oil, and combinations thereof.

In certain embodiments, the dosage form is formulated as granules, pellets, micro particles, tablet, hard shell capsules, suspended in a liquid, suspended in a syrup or enema. In certain embodiments, the dosage form is formulated for oral or mucosal delivery. In certain embodiments, the dosage form is formulated as or in a lozenge, candy, gummy, chocolate or cookie. In certain embodiments, the tablet or pellets are an immediate release or slow or controlled release dosage forms, extended-release forms, or sustained release forms. In certain embodiments the tablet is enteric coated or is a melt or dissolved in the mouth or is muco-adhesive dosage form.

In certain embodiments, the unit dosage form which is a unit particles, such as tablet, capsule, granules, pellets, micro-particles and film, are enteric coated or coated with a colonic coat that protect the unit dose from being decomposed at the acidic gastric pH and swells in time manner of pH controlled manner or both, to release the CBD formulation at the distal intestine and may also release part of the cannabinoids in the intestines for systemic absorption and part of the cannabinoids at the colon for local colonic pharmacological effect.

In certain embodiments, the caffeine, tart cherry, citicoline formulation is formulated in a semi solid or liquid dosage form such as cream, lotion, ointment, dispersion, suspension, gel, foam, spray, syrup, liquid, eye drops, ear drops, enema or an oral dosage form or a topical dosage form or a local ophthalmic or optic or oral cavity or vaginal or rectal or uterine dosage form. Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g., by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

In certain embodiments, any one of the compositions described above, or any one of the dosage forms described above, is for use in a method of treating sleep symptoms or disorders.

Preferred dosage forms include, but are not limited to, any liquid or semi solid or solid dosage form. The caffeine, tart cherry, citicoline formulation may be formulated in a medicament by preparing a topical or mucosal or oral delivery system. The topical delivery system may be in the form of eye drops, a suspension, ointment, cream, foam, spray, topical patch. The oral delivery system may be a tablet or capsule or soft capsule or sachet or granules or a syrup. The mucosal delivery system may be a gel, pessary, enema, douche, wash, foam, mucoadhesive gel or tablet for immediate or for slow or controlled release. The vehicle may comprise any acceptable solvent and inactive ingredients as well as preservatives antioxidants and coloring agents. The delivery form may be single dose or multiple doses as well as micro particle granulate nanoparticle microcapsule liposome micelle, and the like as known in the art of pharmaceutical, cosmetic, veterinary medicine and art of formulation. Further details of suitable dosage forms may be obtained from any standard reference work in this field, including, for example: Remington's Pharmaceutical Sciences, Mack Publishing Co, Easton, Pa, USA (1980).

Thus, in some embodiments of the present invention, the caffeine, tart cherry, citicoline formulation further comprises one or more excipients selected from the group consisting of solvents, stabilizers, suspending agents, emulsifiers, release modifying, targeting and viscosity agents and combinations thereof.

### Preparation

The manufacture of the gummy compositions of the invention involves well known processes. The components of the gummy are typically combined at elevated temperature, i.e., above the temperature at which the gelating agent will gel. Often warm water (e.g., 60 to 80′C water) will be used to ensure that the components, in particular the gelating agents, are in liquid form at this point. What is formed is therefore a solution of the components (although some particulates e.g., from the fruit juice may remain).

Once mixed, the gummy composition can be formed into molds and cooled to allow gelling to occur. The formation of gummy compositions is therefore a well-known process, and it does not need further explanation here. It is the same process used at home to make Jell-O: hot water melts the gelating agent(s), blending of ingredients occurs in the molten gelating agent and gelation is encouraged via cooling.

It may be that the fruit juice can act as a source of most of the water needed to prepare the gummy composition. A person skilled in the art can devise suitable manufacturing processes.

### Example

Participants receiving the citicoline-caffeine beverage exhibited increased performance on tasks associated with mental alertness, attention, and working memory in problem solving compared to a placebo condition (Bruce, 2012). Specifically, the citicoline-caffeine group displayed significantly faster maze learning times, fewer false alarm errors in a Go No-Go task measuring sustained cognitive effort, faster reaction times on a continuous performance test, and better accuracy on a measure of visual spatial processing speed. Bruce S E, Werner K B, Preston B F, Baker L M. Improvements in concentration, working memory and sustained attention following consumption of a natural citicoline-caffeine beverage. Int J Food Sci Nutr. 2014 December; 65(8):1003-7. 2014 Jul. 21. Moreover, examination of ERP indices revealed significantly larger P450 amplitudes in the citicoline group, particularly in pre-frontal and frontal areas which have been associated with working memory and sustained attention (Hermens et al., 2005; Mangina, Beuzeron-Mangina, & Grizenko, 2000; Williams et al., 2010). Evidence of improved P450 amplitude indicates a general improvement in the ability to accommodate new and relevant information within working memory and overall enhanced brain activity.

Results from this example suggests citicoline may play a role in increasing attention and alertness (Babb et al., 2002; Bruce, 2012; Cotroneo et al., 2013; Silveri et al., 2008). Citicoline is a compound that consists of cytidine and choline. It has been shown to activate the biosynthesis of phospholipids (PDE) in neuronal membranes (Babb et al., 2002). Since PDE has been shown to decrease with age and be associated with cognitive memory loss, increase in this substance may improve overall cognitive skills. In a recent study examining mild cognitive impairment in 349 elderly patients, Cotroneo and colleagues (2013) found that the citicoline group showed improvement in mini mental state examination (MMSE) scores after 9 months, compared to the untreated group that showed significant MMSE declines over the same time period. Other investigators have also found that increases in PDE from citicoline supplements have led to improved memory in older adults (Babb et al., 2002). Silveri et al. (2008) conclude that citicoline may assist in reducing cognitive declines associated with aging.

In addition to citicoline, a voluminous body of research supports the biological effects of caffeine on attention and executive functioning. In a recent review of caffeine's effects on attention, Einother and Giesbrecht (2013) concluded that caffeine has substantial benefits in improving both simple and complex attention tasks. Recent functional magnetic resonance imaging (fMRI) studies have also shown beneficial results with respect to caffeine and attentional processes. In a study examining the effects of caffeine in a sample of 24 healthy elderly participants, Haller et al. (2013) found that acute caffeine intake increases activity level in specific brain regions associated with working memory. Koppelstatter et al. (2008) also found that caffeine increases fMRI signal changes in a network of brain areas associated with executive and attentional functions during working memory processes. Both caffeine and citicoline are substances that may play a role in improving memory performance by increasing expression of a sodium potassium pump enzyme (Na+, K+-ATPase), in which deficits have been associated with Alzheimer's disease (Zhang et al. (2013). As such, a possible synergistic effect of citicoline and caffeine may exist. Few studies of caffeine alone have integrated these electrophysiological markers and cognition in the same study using this dose of caffeine. As such, even if driven by caffeine alone, the literature provides new information regarding stimulant effects on multifaceted markers of brain function.

### Conclusions

Results of the present example show that 250 mg of citicoline, when combined with caffeine, results in significant improvements in measures of sustained attention and working memory. Studies with an older adult sample may assist in the generalizability of these findings to an aging population. Moreover, the specific effects of each of the ingredients alone (citicoline, caffeine) compared with the other active compounds in the beverage were not systematically examined and thus, discrimination the single effects of each substance could not be ascertained. However, results support and enable a citicoline-caffeine based beverage is associated with improvements in measures of attention and mental alertness.

### REFERENCES

Arnett J A, Labovitz S S. Effect of physical layout in performance of the Trail Making Test. Psychological Assessment. 1995;7:220-221.
Babb S M, Wald L L, Cohen B M, Villafuerte R A, Gruber S A, Yurgelun-Todd D A, Renshaw P F. Chronic citicoline increases phosphodiesters in the brains of healthy older subjects: an in vivo phosphorus magnetic resonance spectroscopy study. Psychopharmacology (Berl) 2002;161:248-254.
Barker R G. The stepping-stone maze: A directly visible space-problem apparatus. Journal of General Psychology. 1931;5:280-285.
Bruce S E. Improvements in quantitative EEG following consumption of a natural citicoline-enhanced beverage. International Journal of Food Sciences and Nutrition. 2012;63(4):421-425.
Clark C R, Orr R S, Wright E K, Weber D L. Brain Topography Today. Tokyo: Elsevier Science; 1998. Working memory updating to visual verbal stimuli: a high resolution ERP study; pp. 173-178.
Conant R, Schauss A G. Therapeutic applications of citicoline for stroke and cognitive dysfunction in the elderly: a review of the literature. Altern Med Rev. 2004;9:17-31.
Cotroneo A M, Castagna A, Putignano S, Lacava R, Fantò F, Monteleone F, Rocca F, Malara A, Gareri P. Effectiveness and safety of citicoline in mild vascular cognitive impairment: the IDEALE study. Clin Interv Aging. 2013;8:131-137. [PMC free article]
Crowe S F, Barclay L, Brennan S, Farkas L, Gould E, Katchmarsky S, Vayda S. The cognitive determinants of performance on the Austin Maze. Journal of the International Neuropsychological Society. 1999;5(1):1-9.
Einöther S J, Giesbrecht T. Caffeine as an attention enhancer: reviewing existing assumptions. Psychopharmacology (Berl) 2013;225:251-274.
Gatti G, Barzaghi N, Acuto G, Abbiati G, Fossati T, Perucca E. A comparative study of free plasma choline levels following intramuscular administration of L-alpha-glycerylphosphorylcholine and citicoline in normal volunteers. International journal of clinical pharmacology, therapy, and toxicology. 1992;30(9): 331.
Haller S, Rodriguez C, Moser D, Toma S, Hofmeister J, Sinanaj I, Van De Ville D, Giannakopoulos P, Lovblad K O. Acute caffeine administration impact on working memory-related brain activation and functional connectivity in the elderly: a BOLD and perfusion MRI study. Neuroscience. 2013;250:364-371.
Hermens D F, Williams L M, Clarke S, Kohn M, Cooper N, Gordon E. Responses to methylphenidate in adolescent AD/HD: Evidence from concurrently recorded autonomic (EDA) and central (EEG and ERP) measures. Int J Psychophysiol. 2005;58:21-33.
Hocking J, Thomas H J, Dzafic I, Williams R J, Reutens D C, Spooner D M. Disentangling the cognitive components supporting austin maze performance in left versus right temporal lobe epilepsy. Epilepsy & Behavior. 2013
Huang-Pollock C L, Karalunas S L, Tam H, Moore A N. Evaluating vigilance deficits in ADHD: A meta-analysis of CPT performance. Journal of abnormal psychology. 2012;121(2):360. [PMC free article]
Iaboni F, Doublas V I, Baker A G. Effects of reward and response costs on inhibition in ADHD children. Journal of Abnormal Psychology. 1995;104(1):232-240.
Keage H A, Clark C R, Hermens D F, Williams L M, Kohn M R, Clarke S, Gordon E. ERP indices of working memory updating in AD/HD: differential aspects of development, subtype, and medication. Journal of Clinical Neurophysiology. 2008;25(1):32-41.
Koppelstaetter F, Poeppel T D, Siedentopf C M, Ischebeck A, Verius M, Haala I, Mottaghy F M, Rhomberg P, Golaszewski S, Gotwald T, Lorenz I H, Kolbitsch C, Felber S, Krause B J. Does caffeine modulate verbal working memory processes? An fMRI study. Neuroimage. 2008;39:492-499.
Lezak M D, Howieson D B, Bigler E D, Tranel D. Neuropsychological Assessment. 5th ed. New York: Oxford; 2012.
Mangina C A, Beuzeron-Mangina J, Grizenko N. Event-related brain potentials, bilateral electrodermal activity and Mangina-Test performance in learning disabled ADHD pre-adolescents with severe behavioral disorders as compared to age-matched normal controls. International Journal of Psychophysiology. 2000;37:71-85.
Milner B. Visually-guided maze learning in man: Effects of bilateral hippocampal, bilateral frontal, and unilateral cerebral lesions. Neuropsychologia. 1965;3(4):317-338. doi: http://dx.doi.org/10.1016/0028-3932(65)90005-9.
Perry R J, Hodges J R. Attention and executive deficits in Alzheimer's disease: A critical review. Brain. 1999;122:383-404.
22. Riccio C A, Reynolds C R, Lowe P, Moore J J. The continuous performance test: a window on the neural substrates for attention? Archives of clinical neuropsychology. 2002;17(3):235-272.
Riccio C A, Waldrop J J M, Reynolds C R, Lowe P. Effects of stimulants on the continuous performance test (CPT): Implications for CPT use and interpretation. Journal of Neuropsychiatry and Clinical Neurosciences. 2001;13:326-335.
Rossi M, Zanardi M. An open study on the clinical efficacy of citicoline in patients with chronic cerebral vasculopathy. Clin Ter. 1993;142:141-144.
Secades J J. Citicoline: pharmacological and clinical review, 2010 update. Rev Neurol. 2011;14(Suppl 2):S1-S62.
Secades J J, Lorenzo J L. Citicoline: pharmacological and clinical review, 2006 update. Methods Find Exp Clin Pharmacol. 2006;28(Suppl B):1-56.
Silveri M M, Dikan J, Ross A J, Jensen J E, Kamiya T, Kawada Y, Yurgelun-Todd D A. Citicoline enhances frontal lobe bioenergetics as measured by phosphorus magnetic resonance spectroscopy. NMR in Biomedicine. 2008;21(10):1066-1075.
Weafer J, Baggott M J, de Wit H. Test-Retest reliability of behavioral measures of impulsive choice, impulsive action, and inattention. Experimental and Clinical Psychopharmacology. 2013 Retrieved from. [PMC free article]
Wechsler D. Wechsler Adult Intelligence Scale—Revised: Manual. New York: Psychological Corporation; 1981.
Williams L M, Hermens D F, Thein T, Clark C R, Cooper N J, Clarke S D, Kohn M R. Using brain-based cognitive measures to support clinical decisions in ADHD. Pediatric Neurology. 2010;42(2):118-126.
Zhang L N, Sun Y J, Pan S, Li J X, Qu Y E, Li Y, Wang Y L, Gao Z B. Na+-K+-ATPase, a potent neuroprotective modulator against Alzheimer disease. Fundam Clin Pharmacol. 2013;27:96-103.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of providing a synergistic effect of caffeine with decreasing the negative side effects of caffeine and increasing the energy provided, comprising: administering an effective amount of caffeine, an effective amount of tart cherry extract, an effective amount of citicoline in a composition or formulation, wherein the effective amount of caffeine is in a controlled-release format to exert its effects and the effective amount of caffeine synergistically effects tart cherry extract and citicoline immediately and over a period of at least 4 hours to provide the benefits of caffeine without the negative effects, wherein the tart cherry extract includes antioxidant support from caffeine and citicoline both immediately and over an extended period of time.

2. The method of claim 1, wherein the composition or formulation is to be ingested as a dietary supplement, functional food or functional beverage.

3. The method of claim 1, wherein the effective amount of caffeine, citicoline, and tart cherry extract synergistically provides an immediate and sustained for Energy, Nootropic effect, Mental and physical performance, Recovery, or Antioxidant support.

4. The method of claim 3, wherein the effective amount of caffeine is between about 1 mg and about 600 mg per serving.

5. The method of claim 4, wherein the effective amount of Citicoline is between about 1 mg and about 1,000 mg per serving.

6. The method of claim 5, wherein the effective amount of Tart Cherry Extract is between about 1 mg and about 1000 mg per serving.

7. The method of claim 6, wherein caffeine comprises a caffeine derivative, a caffeine substitute including Alpinia galanga, or a caffeine modifier.

8. A composition or formulation comprising: an effective amount of caffeine, an effective amount of tart cherry extract, an effective amount of citicoline, wherein the effective amount of caffeine is in a controlled-release format to exert its effects and the effective amount of caffeine synergistically effects tart cherry extract and citicoline immediately and over time.

9. The composition or formulation of claim 8, wherein the controlled release of caffeine is over a period of at least 4 hours to provide the benefits of caffeine without the negative effects.

10. The composition or formulation of claim 9, wherein the tart cherry extract includes antioxidant support from caffeine and citicoline both immediately and over an extended period of time.

11. The composition or formulation of claim 10, wherein the composition or formulation is to be ingested as a dietary supplement, functional food or functional beverage.

12. The composition or formulation of claim 11, wherein the effective amount of caffeine, citicoline, and tart cherry extract synergistically provides an immediate and sustained for Energy, Nootropic effect, Mental and physical performance, Recovery, or Antioxidant support.

13. The composition or formulation of claim 12, wherein the effective amount of caffeine is between about 1 mg and about 600 mg per serving.

14. The composition or formulation of claim 13, wherein the effective amount of Citicoline is between about 1 mg and about 1,000 mg per serving.

15. The composition or formulation of claim 14, wherein the effective amount of Tart Cherry Extract is between about 1 mg and about 1000 mg per serving.

16. The composition or formulation of claim 15, wherein caffeine comprises a caffeine derivative, a caffeine substitute including Alpinia galanga, or a caffeine modifier.

17. A composition or formulation comprising: an effective amount of caffeine, an effective amount of an antioxidant, an effective amount of citicoline, wherein the effective amount of caffeine is in an immediate-release format to exert its effects and the effective amount of caffeine synergistically effects the antioxidant and citicoline immediately and over time.

18. The composition or formulation of claim 17, wherein the controlled release of caffeine is over a period of at least 4 hours to provide the benefits of caffeine without the negative effects.

19. The composition or formulation of claim 18, wherein the antioxidant includes antioxidant support from caffeine and citicoline both immediately and over an extended period of time.

20. The composition or formulation of claim 19, wherein the composition or formulation is to be ingested as a dietary supplement, functional food or functional beverage.
